(19) United States
(12) Patent Application Publication
(10) Pub. No.: US 2026/0000896 A1
(43) Pub. Date: Jan. 1, 2026

(54) NEUROSTIMULATION BLOCK SEQUENCES WITH RAMPING

(71) Applicant: Boston Scientific Neuromodulation Corporation, Valencia, CA (US)

(72) Inventor: Sarvani Grandhe, Valencia, CA (US)

(21) Appl. No.: 19/250,713

(22) Filed: Jun. 26, 2025

Related U.S. Application Data
(60) Provisional application No. 63/666,422, filed on Jul. 1, 2024.

(51) Int. Cl. A61N 1/36; A61N 1/372

(52) U.S. Cl. CPC A61N 1/36139; A61N 1/36125; A61N 1/36196; A61N 1/37247; A61N 1/37252

(57) ABSTRACT

A system may include a neurostimulator and a processing system. The neurostimulator may be configured to provide a neurostimulation field by delivering electrical energy according to a set of values for a stimulation parameter set including adjustable parameter(s). The processing system may be configured to create a sequence of blocks to determine the neurostimulation field. The sequence may include stimulation program block(s) and ramp block(s) configured to determine a ramping sequence for changing the parameter values for the adjustable parameter(s). The ramp block(s) may be configured to determine a ramping sequence for changing the parameter values for the adjustable parameter(s), and may include a preceding ramp block and/or a subsequent ramp block. The neurostimulator is configured to provide the neurostimulation field according to the sequence of blocks.

### CLAIM OF PRIORITY

This application claims the benefit of U.S. Provisional Application No. 63/666,422, filed on Jul. 1, 2024, which is hereby incorporated by reference in its entirety.

### TECHNICAL FIELD

This document relates generally to medical devices, and more particularly, to systems, devices and methods for providing neurostimulation using block sequences.

### BACKGROUND

Examples of neurostimulation therapy devices include, but are not limited to, transcutaneous electrical neural stimulators (TENS), spinal cord stimulators (SCS), cortical and Deep Brain Stimulators (DBS), Peripheral Nerve Stimulation (PNS), and Functional Electrical Stimulation (FES). A therapy device may be configured or programmed to treat a condition. Thus, by way of example and not limitation, a DBS system may be configured to treat motor disorders such as, but not limited to, tremor, bradykinesia, and dyskinesia associated with Parkinson's Disease (PD). In another nonlimiting example, a stimulation device, such as neurostimulation device (e.g., DBS, SCS, PNS or TENS), may be configured to treat pain. Settings of the therapy device, including stimulation parameters, may be programmed to provide desirable intended effects (e.g., reduced tremor, bradykinesia, and dyskinesia for a PD therapy, desirable pain relief or paresthesia coverage for a pain therapy) while avoiding undesirable side effects. Stimulation parameters may be used to provide a therapy using patterns of neurostimulation.

### SUMMARY

This disclosure provides an improved system and process for programming neurostimulation patterns. A stimulation program may be composed to include a programmable temporal order of stimulation program blocks to create a neurostimulation pattern. Preceding ramp blocks and/or successive ramp blocks may be inserted into the sequence to assist with a transition to and/or from a stimulation program block. Such ramp blocks may improve the comfort for delivering the neurostimulation defined by a sequence of stimulation program blocks and may enable more options for creating the sequence of stimulation program blocks. By way of example and not limitation, ramp blocks may enable larger amplitude stimulation to be used in the sequence and/or may enable adjacent blocks to have larger differences in amplitude.

An example (e.g., “Example 1”) of a system may include a neurostimulator and a processing system. The neurostimulator may be configured to provide a neurostimulation field by delivering electrical energy according to a set of values for a stimulation parameter set. The stimulation parameter set may include adjustable parameter(s). The processing system may be configured to create a sequence of blocks to determine the neurostimulation field provided by the neurostimulator. The sequence of blocks may include stimulation program block(s) configured to provide the neurostimulation field by delivering electrical energy according to the set of values for the stimulation parameter set, and ramp block(s) configured to determine a ramping sequence for changing the parameter values for the adjustable parameter(s). The set of values may include program value(s) for the adjustable parameter(s). The ramp block(s) may be configured to determine a ramping sequence for changing the parameter values for the adjustable parameter(s), including at least one of a preceding ramp block configured to change parameter values for the adjustable parameter(s) to ramp toward the program value(s) or a subsequent ramp block configured to change the parameter values for the adjustable parameter(s) to ramp away from the program value(s). The neurostimulator is configured to provide the neurostimulation field according to the sequence of blocks.

In Example 2, the subject matter of Example 1 may optionally be configured such that the adjustable parameter(s) include at least one of a pulse amplitude, a pulse width, a pulse-to-pulse frequency, a pulse train duration, a burst frequency, or a dose parameter.

In Example 3, the subject matter of Example 2 may optionally be configured such that the adjustable parameter(s) includes the pulse amplitude and the ramp block(s) only adjusts the pulse amplitude.

In Example 4, the subject matter of any one or more of Examples 1-3 may optionally be configured such that the stimulation program block includes more than one pulse and each of the adjustable parameter(s) has one value.

In Example 5, the subject matter of any one or more of Examples 1-3 may optionally be configured such that the stimulation program block includes more than one pulse and one or more of the adjustable parameters have more than one value.

In Example 6, the subject matter of any one or more of Examples 1-3 may optionally be configured such that the stimulation program block includes one pulse and the adjustable parameter(s) includes at least one of a pulse amplitude or a pulse width for the one pulse.

In Example 7, the subject matter of any one or more of Examples 1-6 may optionally be configured such that the processing system is configured to automatically insert the ramp block(s) into the sequence of blocks based on the program value(s) for the adjustable parameter(s) in the stimulation program block(s).

In Example 8, the subject matter of any one or more of Examples 1-7 may optionally be configured such that the processing system includes a user interface configured to receive user input and the processing system is configured to insert the ramp block(s) based on the received user input.

In Example 9, the subject matter of any one or more of Examples 1-8 may optionally be configured such that the ramp block(s) includes a sequence of three pulses corresponding to three parameter values for the adjustable parameter(s).

In Example 10, the subject matter of any one or more of Examples 1-9 may optionally be configured such that the ramp block(s) includes a sequence of at least two pulses corresponding to one parameter value for the adjustable parameter(s).

In Example 11, the subject matter of any one or more of Examples 1-10 may optionally be configured such that the processing system further includes a ramp control interface configured to enable user control of the ramping sequence when the neurostimulator is providing the neurostimulation field according to the ramping sequence.

In Example 12, the subject matter of Example 11 may optionally be configured such that the ramp control interface includes at least one of a stop element to provide a stop command to the processing system to stop the ramping sequence, a pause element to provide a pause command to the processing system to pause the ramping sequence, a play element to provide a play command to the processing system to normally progress through the ramping sequence, a skip element to provide a skip command to the processing system to skip to another value in the ramping sequence, a skip ramp element to provide a skip ramp command to the processing system to skip to an end of the ramping sequence, or a speed control to provide a speed command to the processing system to change a rate for progressing through the ramping sequence.

In Example 13, the subject matter of any one or more of Examples 1-12 may optionally be configured such that the processing system further includes a ramp parameter selection interface configured to enable user selection of the adjustable parameter(s) in the ramp block(s) from at least two available adjustable parameters. The available adjustable parameters include two or more of a pulse amplitude, a pulse width, a pulse-to-pulse frequency, a pulse train duration, a burst frequency, or a dose.

In Example 14, the subject matter of any one or more of Examples 1-13 may optionally be configured such that the processing system further includes a ramp configuration interface configured to enable user configuration of the ramping sequence, including at least one of a start value for the ramping sequence, an end value for the ramping sequence, a number of ramp levels, a duration for at least some of the ramp levels, values for at least some of the ramp levels, an overall ramp duration, or a ramp shape.

In Example 15, the subject matter of Example 14 may optionally be configured such that the ramp configuration interface is configured to enable user configuration of the ramp shape for the ramping sequence, including user configuration of a non-linear ramp shape.

Example 16 includes subject matter (such as a method, means for performing acts, machine readable medium including instructions that when performed by a machine cause the machine to perform acts, or an apparatus to perform). The subject matter may include creating a sequence of blocks to determine a neurostimulation field provided by a neurostimulator. The sequence of blocks may include stimulation program block(s) and ramp block(s). The stimulation program block(s) may be configured to provide the neurostimulation field by delivering electrical energy according to a set of values for a stimulation parameter set that includes adjustable parameter(s). The set of values includes program value(s) for the adjustable parameter(s). The ramp block(s) may include a preceding ramp block configured to change parameter values for the adjustable parameter(s) to ramp toward the program value(s) or a subsequent ramp block configured to change the parameter values for the adjustable parameter(s) to ramp away from the program value(s). The subject matter may include programming the neurostimulator to provide the neurostimulation field according to the sequence of blocks.

In Example 17, the subject matter of Example 16 may optionally be configured such that the adjustable parameter(s) include at least one of a pulse amplitude, a pulse width, a pulse-to-pulse frequency, a pulse train duration, a burst frequency, or a dose parameter.

In Example 18, the subject matter of Example 17 may optionally be configured such that the adjustable parameter(s) includes the pulse amplitude and the ramp block(s) only adjusts the pulse amplitude.

In Example 19, the subject matter of any one or more of Examples 16-18 may optionally be configured such that the stimulation program block includes more than one pulse and each of the adjustable parameter(s) has one value.

In Example 20, the subject matter of any one or more of Examples 16-18 may optionally be configured such that the stimulation program block includes more than one pulse and one or more of the adjustable parameter(s) has more than one value.

In Example 21, the subject matter of any one or more of Examples 16-18 may optionally be configured such that the stimulation program block includes one pulse and the adjustable parameter(s) includes at least one of a pulse amplitude or a pulse width for the one pulse.

In Example 22, the subject matter of any one or more of Examples 16-21 may optionally be configured such that the creating the sequence of blocks includes automatically inserting the ramp block(s) into the sequence of blocks based on the program value(s) for the adjustable parameter(s) in the stimulation program block(s).

In Example 23, the subject matter of any one or more of Examples 16-22 may optionally be configured such that the creating the sequence of blocks includes receiving user input and inserting the ramp block(s) based on the received user input.

In Example 24, the subject matter of any one or more of Examples 16-23 may optionally be configured such that the ramp block(s) includes a sequence of three pulses corresponding to three parameter values for the adjustable parameter(s).

In Example 25, the subject matter of any one or more of Examples 21-24 may optionally be configured such that the ramp block(s) includes a sequence of at least two pulses corresponding to one parameter value for the adjustable parameter(s).

In Example 26, the subject matter of any one or more of Examples 16-25 may optionally be configured to further include using the neurostimulator to provide the neurostimulation field according to a ramping sequence and providing a ramp control interface to enable user control of the ramping sequence when the neurostimulator is providing the neurostimulation field according to the ramping sequence.

In Example 27, the subject matter of Example 26 may optionally be configured such that the ramp control interface includes at least one of a stop element to provide a stop command to a processing system to stop the ramping sequence, a pause element to provide a pause command to the processing system to pause the ramping sequence, a play element to provide a play command to the processing system to normally progress through the ramping sequence, a skip element to provide a skip command to the processing system to skip to another value in the ramping sequence, a skip ramp element to provide a skip ramp command to the processing system to skip to an end of the ramping sequence, or a speed control to provide a speed command to the processing system to change a rate for progressing through the ramping sequence.

In Example 28, the subject matter any one or more of claims 16-27 may optionally be configured to include providing a ramp parameter selection interface configured to enable user selection of the adjustable parameter(s) in the ramp block(s) from at least two available adjustable parameters. The available adjustable parameters include two or more of a pulse amplitude, a pulse width, a pulse-to-pulse frequency, a pulse train duration, a burst frequency, or a dose.

In Example 29, the subject matter of any one or more of Examples 16-29 may optionally be configured to further include providing a ramp configuration interface configured to enable user configuration of the ramping sequence, including at least one of a start value for the ramping sequence, an end value for the ramping sequence, a number of ramp levels, a duration for at least some of the ramp levels, values for at least some of the ramp levels, an overall ramp duration, or a ramp shape.

In Example 30, the subject matter of Example 29 may optionally be configured such that the ramp configuration interface is configured to enable user configuration of the ramp shape for the ramping sequence, including user configuration of a non-linear ramp shape.

Example 31 includes subject matter that includes non-transitory machine-readable medium including instructions, which when executed by a machine, cause the machine to perform a method. The method may include, by way of example and not limitation, any of the subject matter for one or more of Examples 16-30. The machine-readable medium may include instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine-readable medium” is intended to include at least one machine-readable medium (e.g., two or more media which may be of the same type of media (such as but not limited to different nonvolatile semiconductor memory arrays) or different type of media (such as but not limited to a hard disk and a non-volatile semiconductor memory array). Furthermore, the term “machine” may include at least one processor, including one processor to implement all of the instructions, at least two processors where one processor operates on some of the instructions and other processor(s) operate on other instructions, or at least two processors where each processor is capable of operating on the same instructions. Thus, for example, distributed systems or systems with shared resources are contemplated.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various examples are illustrated by way of example in the figures of the accompanying drawings. Such examples are demonstrative and not intended to be exhaustive or exclusive examples of the present subject matter.

FIG. 1 illustrates an example of an electrical stimulation system.

FIG. 2 illustrates an example of an implantable pulse generator (IPG) that may be used in a DBS system.

FIGS. 3A-3B illustrate examples of leads that may be coupled to an IPG to deliver electrostimulation such as DBS.

FIG. 4 illustrates an example of a computing device or system for programming or controlling the operation of an electrostimulation system.

FIG. 5 illustrates an example of a stimulation parameter control system and a part of the environment in which it may operate.

FIG. 6 illustrates, by way of example, an example of an electrical therapy-delivery system.

FIG. 7 illustrates, by way of example and not limitation, an implantable electrical therapy-delivery system.

FIG. 8 illustrates, by way of example and not limitation, an embodiment of a stimulation programming circuit for use in a neurostimulation system.

FIG. 9 illustrates, by way of example and not limitation, an embodiment of a system for delivering neurostimulation such as a DBS therapy.

FIG. 10 illustrates, by way of example and not limitation, block sequences for four timing channels.

FIG. 11 illustrates, by way of example and not limitation, block sequences for different channels with illustrated timing relationships between the blocks in those sequences.

FIG. 12 illustrates, by way of example and not limitation, a system configured to deliver neurostimulation according to sequences of blocks.

FIG. 13 illustrates, by way of example and not limitation, a stimulation program block with a preceding ramp block for ramping toward the stimulation program block and with a subsequent ramp block for ramping away from the stimulation program block.

FIG. 14 illustrates, by way of example and not limitation, a sequence of blocks including stimulation program blocks and ramp blocks.

FIG. 15 illustrates, by way of example and not limitation, a sequence of blocks including stimulation program blocks and preceding and subsequent ramp blocks.

FIG. 16 illustrates, by way of example and not limitation, pulse-to-pulse variations that may be implemented in a ramp block.

FIG. 17 illustrates, by way of example and not limitation, a user interface for programming a sequence of ramp blocks.

FIG. 18 illustrates, by way of example and not limitation, features that may be implemented in at least one user interface.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter provides features for programming neurostimulation patterns. Advancements in neuroscience and neurostimulation research have led to a demand for using complex and/or individually optimized patterns of neurostimulation energy for various types of therapies. A neurostimulation pattern may be custom-defined using building blocks (e.g., “stimulation program blocks”). For example, potentially complex patterns may be defined by creating and editing graphical representations of relatively simple individual building blocks for each of the patterns.

The present subject matter may provide a transition to a stimulation block, at transition from a stimulation block, or a transition between stimulation blocks. These transitions may improve the patient experience by making the transitions more comfortable, which may improve patient acceptance and compliance with the therapy. Further, if the transitions are more comfortable, a larger range of potential stimulation program blocks may be considered that would otherwise not have been considered because of the potential patient discomfort. Thus, for example, ramp blocks may enable larger amplitude stimulation to be used in the sequence and/or may enable adjacent blocks to have larger differences in amplitude. The present subject matter is not limited to amplitude as other stimulation parameters may be ramped.

A user may drag and drop or manually add specific ramp blocks in between stimulation pulses or patterns. Each ramp block can be programmed to have an independent dynamic scaling/speed or step size at which the ramp occurs. A user may stop or pause or change the speed of an ongoing ramp block.

A ramp block may be automatically added based on an amplitude threshold. This automatic feature may be enabled or disabled. The ramp blocks or the ramp modulation parameters/characteristics may be stored in a database used by a programmer or may be stored in the implant, to allow for patient specific ramping. The ramp may be implemented in the software or through the firmware of the stimulator through look up table methods.

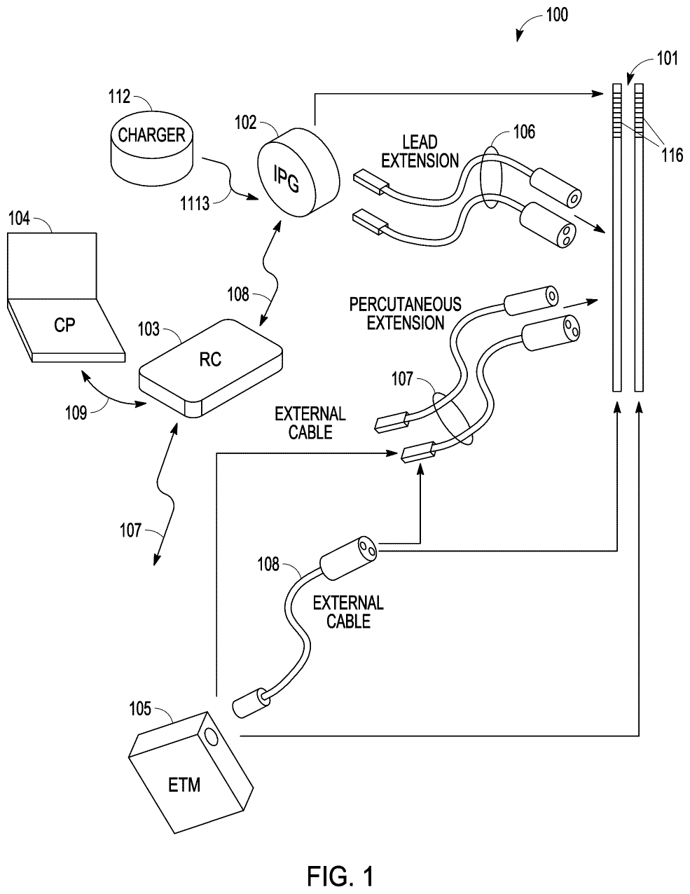

FIG. 1 illustrates, by way of example and not limitation, an electrical stimulation system 100. The electrical stimulation system may be used to deliver DBS or SCS, for example. The electrical stimulation system 100 may generally include a one or more (illustrated as two) of implantable neurostimulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neurostimulation lead(s) 101, which carry a plurality of electrodes 116. The electrodes, when implanted in a patient, form an electrode arrangement. As illustrated, the neurostimulation leads 101 may be percutaneous leads with the electrodes arranged in-line along the neurostimulation leads or about a circumference of the neurostimulation leads. Any suitable number of neurostimulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical stimulation energy in the form of a pulsed electrical waveform (i.e., a temporal series of electrical pulses) to the electrodes in accordance with a set of stimulation parameters.

The ETM 105 may also be physically connected via the percutaneous lead extensions 107 and external cable 108 to the neurostimulation lead(s) 101. The ETM 105 may have similar pulse generation circuitry as the IPG 102 to deliver electrical stimulation energy to the electrodes in accordance with a set of stimulation parameters. A programming process may be used to test different parameter sets. The ETM 105 is a non-implantable device that may be used on a trial basis after the neurostimulation leads 101 have been implanted and prior to implantation of the IPG 102, to test the responsiveness of the stimulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different stimulation parameter sets. The IPG 102 may also be operated to modify the programmed stimulation parameters to actively control the characteristics of the electrical stimulation energy output by the IPG 102. A clinician may use the CP 104 to program stimulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed stimulation parameters provided by the CP 104 may also be used to program the RC 103, so that the stimulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical stimulation generated by the IPG 102 to allow the desired parameters to be determined based on patient feedback or other feedback and for subsequently programming the IPG 102 with the desired stimulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g. CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), define the configuration and orientation of the leads, initiate and control the electrical stimulation energy output by the neurostimulation leads, and select and program the IPG with stimulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

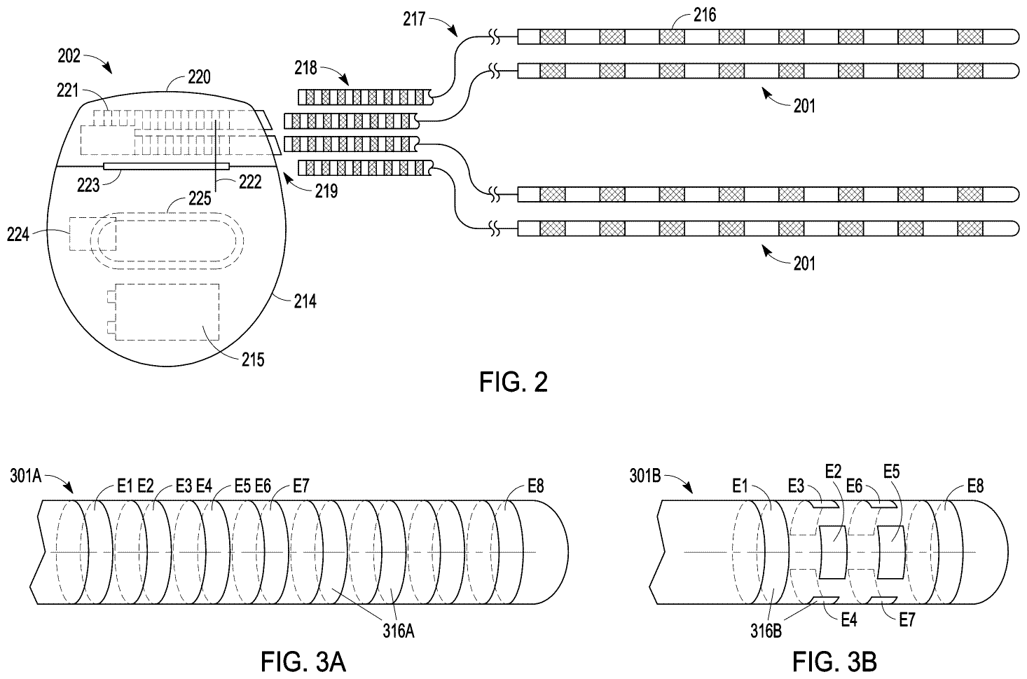

FIG. 2 illustrates, by way of example and not limitation, an IPG 202 that may be used in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 may also lack a battery and may be wirelessly powered by an external source. The IPG 202 may be coupled to one or more leads, such as leads 201 as illustrated herein. The leads 201 may each include a plurality of electrodes 216 for delivering electrostimulation energy, recording electrical signals, or both. In some examples, the leads 201 may be rotatable so that the electrodes 216 may be aligned with the target neurons after the neurons have been located such as based on the recorded signals. The electrodes 216 may include one or more ring electrodes, and/or one or more sets of segmented electrodes (or any other combination of electrodes), examples of which are discussed below with reference to FIGS. 3A and 3B.

The leads 201 may be implanted near or within the desired portion of the body to be stimulated. In an example of operations for DBS, access to the desired position in the brain may be accomplished by drilling a hole in the patient's skull or cranium with a cranial drill (commonly referred to as a burr), and coagulating and incising the dura mater, or brain covering. A lead may then be inserted into the cranium and brain tissue with the assistance of a stylet (not shown). The lead may be guided to the target location within the brain using, for example, a stereotactic frame and a microdrive motor system. In some examples, the microdrive motor system may be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header may comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG may vary according to the application.

The IPG 202 may include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, MICS, and the like.

In a DBS application, as is useful in the treatment of tremor in Parkinson's disease for example, the IPG 202 is typically implanted under the patient's clavicle (collarbone). The leads 201 (which may be extended by lead extensions, not shown) may be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 may also be implanted underneath the scalp closer to the location of the electrodes' implantation. The leads 201, or the extensions, may be integrated with and permanently connected to the IPG 202 in other solutions.

Stimulation in IPG 202 is typically provided by pulses each of which may include one phase or multiple phases. For example, a monopolar stimulation current may be delivered between a lead-based electrode (e.g., one of the electrodes 216) and a case electrode. A bipolar stimulation current may be delivered between two lead-based electrodes (e.g., two of the electrodes 216). Stimulation parameters typically include current amplitude (or voltage amplitude), frequency, pulse width of the pulses or of its individual phases, electrodes selected to provide the stimulation, polarity of such selected electrodes, i.e., whether they act as anodes that source current to the tissue, or cathodes that sink current from the tissue. Each of the electrodes may either be used (an active electrode) or unused (OFF). When the electrode is used, the electrode may be used as an anode or cathode and carry anodic or cathodic current. The anodic energy contributions may be distributed across more than one anode and the cathodic energy contributions may be distributed across more than one cathode (e.g., electrode fractionalization). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. In some instances, an electrode might be an anode for a period of time and a cathode for a period of time. These and possibly other stimulation parameters taken together comprise a stimulation program that the stimulation circuitry 224 in the IPG 202 may execute to provide therapeutic stimulation to a patient.

In some examples, a measurement device coupled to the muscles or other tissue stimulated by the target neurons, or a unit responsive to the patient or clinician, may be coupled to the IPG 202 or microdrive motor system. The measurement device, user, or clinician may indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device may be used to observe the muscle and indicate changes in, for example, tremor frequency or amplitude in response to stimulation of neurons. Alternatively, the patient or clinician may observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 may direct stimulus current to a selected angular range around the lead.

Segmented electrodes may provide better current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering may be performed not only along a length of the lead but also around a circumference of the lead. This provides precise three-dimensional targeting and delivery of the current stimulus to neural target tissue, while potentially avoiding stimulation of other tissue. In some examples, lead(s) may include both segmented electrodes and ring electrodes. A lead which includes at least one or more segmented electrodes may be referred to as a directional lead. In an example, all electrodes on a directional lead may be segmented electrodes. In another example, there may be different numbers of segmented electrodes at different longitudinal positions. Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes. The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead.

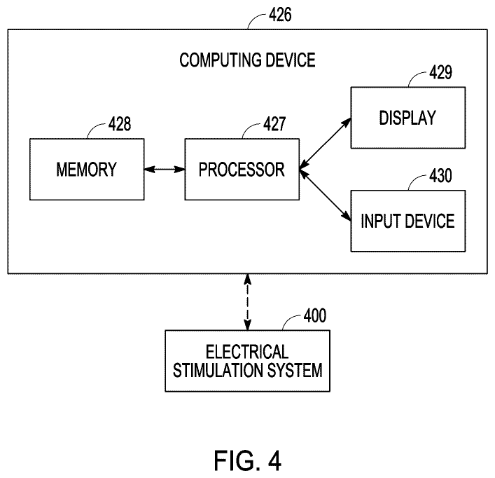

FIG. 4 illustrates, by way of example and not limitation, a computing device or system 426 for programming or controlling the operation of an electrical stimulation system 400. The computing device or system 426 may include a processor 427, a memory 428, a display 429, and an input device 430. Optionally, the computing device or system 426 may be separate from and communicatively coupled to the electrical stimulation system 400, such as system 100 in FIG. 1 Alternatively, the computing device or system 426 may be integrated with the electrical stimulation system 100, such as part of the IPG 102, RC 103, CP 104, or ETM 105 illustrated in FIG. 1.

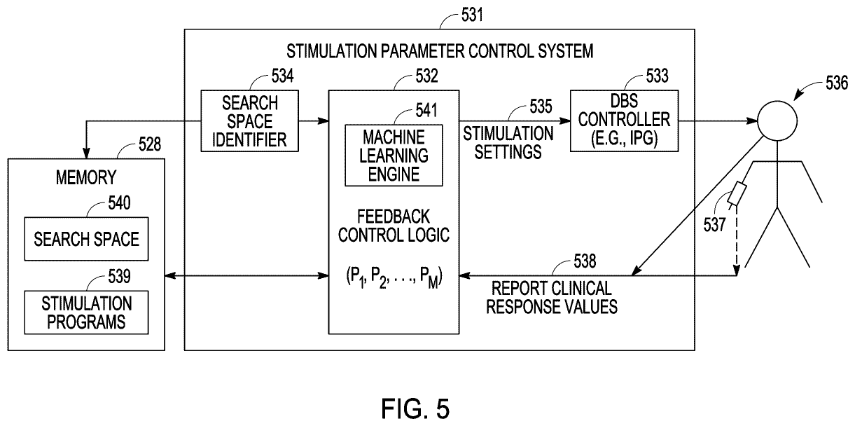

The computing device or system 426, also referred to as a programming device/system or processing device/system, may be a computer, tablet, mobile device, or any other suitable device for processing information. The computing device or system 426 may be local to the user or may include components that are non-local to the computer including one or both of the processor 427 or memory 428 (or portions thereof). For example, the user may operate a terminal that is connected to a non-local processor or memory. In some examples, the computing device or system 406 may include a watch, wristband, smartphone, or the like. Such computing devices/systems may wirelessly communicate with the other components of the electrical stimulation system, such as the CP 104, RC 103, ETM 105, or IPG 102 illustrated in FIG. 1. The computing device or system 426 may be used for gathering patient information, such as general activity level or present queries or tests to the patient to identify or score pain, depression, stimulation effects or side effects, cognitive ability, or the like. In some examples, the computing device or system 426 may prompt the patient to take a periodic test (for example, every day) for cognitive ability to monitor, for example, Alzheimer's disease. In some examples, the computing device or system 426 may detect, or otherwise receive as input, patient clinical responses to electrostimulation such as DBS, and determine or update stimulation parameters using a closed-loop algorithm based on the patient clinical responses, as described below with reference to FIG. 5. Examples of the patient clinical responses may include physiological signals (e.g., heart rate) or motor parameters (e.g., tremor, rigidity, bradykinesia). In some examples, the computing device or system 426 may be a wearable device used by the patient only during programming sessions. Alternatively, the computing device or system 426 may be worn all the time and continually or periodically adjust the stimulation parameters. In an example, the closed-loop algorithm for determining or updating stimulation parameters may be implemented in a mobile device, such as a smartphone, that is connected to the IPG or an evaluating device (e.g., a wristband or watch). These devices may also record and send information to the clinician.

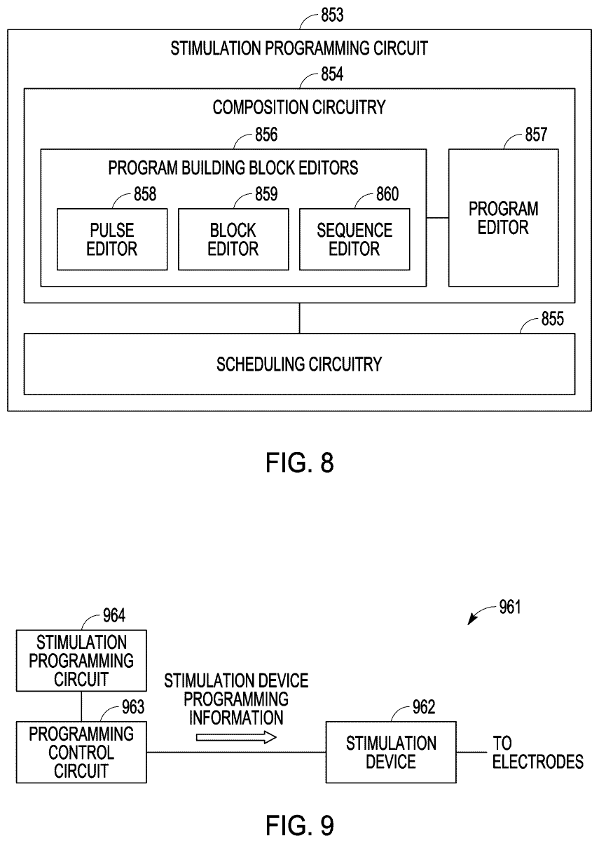

The processor 427 may be implemented as a processing system that include one or more processors that may be local to the user or non-local to the user or other components of the computing device or system 426. In an example, the processor 427 may execute instructions (e.g., stored in the memory 428) to determine a search space of electrode configurations and parameter values, and identify or update one or more stimulation settings that are selectable for use in electrostimulation therapies such as DBS. The search space may include a collection of available electrodes, possible electrode configurations, and possible values or value ranges of one or more stimulation parameters that may be applied to selected electrodes to deliver electrostimulation. The search space may be specific to a particular lead or a type of lead with respect to a specific neural target. As a result, for different leads or types of lead and/or for different neural targets, the processor 427 may determine respective different search spaces. A stimulation setting includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, and like. The stimulation parameters may include stimulation blocks used to created neurostimulation patterns. The use of blocks to create patterns of neurostimulation is discussed in detail below (e.g., discussion of FIGS. 8-18).

The processor 427 may identify or modify a stimulation setting from the search space through an optimization process until a search criterion is satisfied, such as until an optimal, desired, or acceptable patient clinical response is achieved. For example, neurostimulation patterns created from stimulation blocks may be modified until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit may be a dedicated processor such as a digital signal processor, application specific integrated circuit (ASIC), microprocessor, or other type of processor for processing information. Alternatively, the microprocessor circuit may be a processor that may receive and execute a set of instructions of performing the functions, methods, or techniques described herein.

The memory 428 may store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings, and/or instructions for implementing a testing process for testing stimulation parameters. The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, optical storage, magnetic storage, or any other medium which may be used to store the desired information, and which may be accessed by a computing device or system.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth™, near field communication, and other wireless media.

The display 429 may be any suitable display or presentation device, such as a monitor, screen, or the like, and may include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician may observe the patient. Yet another input device 430 may a microphone where the patient or clinician may provide responses or queries.

The electrical stimulation system 400 may include, for example, any of the components illustrated in FIG. 1. The electrical stimulation system 400 may communicate with the computing device or system 426 through a wired or wireless connection or, alternatively or additionally, a user may provide information between the electrical stimulation system 400 and the computing device or system 426 using a computer-readable medium or by some other mechanism.

FIG. 5 illustrates, by way of example and not limitation, a stimulation parameter control system and a part of the environment in which it may operate. The stimulation parameter control system 531, which may be implemented as a part of the processor 427 in FIG. 4, may include a feedback control logic 532, a DBS controller 533, and a search space identifier 534. DBS is used as an example. It is noted that the system may be implemented for other stimulation therapies such as, but not limited to, SCS or PNS. The feedback control logic 532 may be implemented in, for example, the CP 104 or the RC 103 in FIG. 1. The feedback control logic 532 may determine or modify one or more stimulation settings 535 for a stimulation lead at a target stimulation region, such as a region in a brain hemisphere. A stimulation setting may include stimulation patterns such as may be implemented using a sequence of stimulation blocks. A stimulation setting may include an electrode configuration and values for one or more stimulation parameters (P₁, P₂, . . . , Pₘ) 535. The electrode configuration includes information about electrodes (ring electrodes and/or segmented electrodes) selected to be active for delivering stimulation (ON) or inactive (OFF), polarity of the selected electrodes, electrode locations (also referred to as contact locations, which may include longitudinal positions of ring electrodes along the length of a lead, or angular positions of segmented electrodes about a circumference of a cross-section of the lead at a longitudinal position), and stimulation modes (e.g., monopolar pacing or bipolar pacing), etc. The stimulation parameters may include, for example, current amplitude values, current fractionalization across electrodes, stimulation frequency, stimulation pulse width, etc. In some examples, the feedback control logic 532 may modify the stimulation setting 535 such as by changing a stimulation parameter value, modifying an electrode configuration, or modifying neurostimulation patterns.

The stimulation setting 535 may be provided to the DBS controller 533 to configure the IPG or ETM to deliver DBS therapy to the patient 536 in accordance with the stimulation setting or the modified stimulation setting. The stimulation may produce certain therapeutic effects and/or side effects on the patient 536. Such therapeutic effectiveness and side effects, also referred to as clinical responses or clinical metrics, may be provided to the feedback control logic 532. In an example, the clinical responses may be based on patient or clinician observations. For example, motor symptoms such as bradykinesia (slowness of movement), rigidity, tremor, among other symptoms or side effects, may be scored by the patient or by the clinician upon overserving or questioning the patient. In some examples, the clinical responses may be objective in nature, such as measurements automatically or semi-automatically taken by a sensor 537. In an example, the sensor 537 may be included in a wearable device associated with patient 536, such as a smart watch. For example, a Parkinson's patient may be fitted with a wearable sensor that measures tremors, such as by measuring the frequency and amplitude of such tremors.

The clinical responses, either reported by the patient or measured by a sensor, may be converted to clinical response values 538, also referred to as clinical response scores. In an example, the clinical response values 538 may be computed based on the intensity, frequency, or duration of one or more of tremor, rigidity, or bradykinesia responses. Based upon the received clinical response values 538, the feedback control logic 532 may adjust electrode configurations or values of one or more stimulation parameters 535. The feedback control logic 532 may send the adjusted (new or revised) stimulation setting 535, such as the electrode configuration or the adjusted stimulation parameter values, to further configure the DBS controller 533 to change the stimulation parameters of the leads implanted in patient 506 to the adjusted values.

The feedback-control loop as illustrated in FIG. 5 may continue until an optimal, desired, or acceptable outcome is reached, such as maximizing therapeutic effectiveness while minimizing unwanted side effects, or until a specific stop condition is reached such as number of iterations, time spent in programming session, or the like. An outcome may be considered optimal, desired, or acceptable if it meets certain threshold values or tests (e.g., improved clinical response for the patient, faster programming of the device, increased battery life, and/or control multiple independent current sources and directional lead). Such an iterative process of looking for a stimulation setting (e.g., an electrode configuration and stimulation parameter values for the electrode) is referred to as a stimulation setting optimization process. The outcome being reached may be referred to as an optimization criterion, and the resultant stimulation setting may be referred to as an optimal base stimulation setting (BSS). By way of example and not limitation, the optimization criterion may include possible optimal clinical outcome within the parameters chosen; time spent, iterations taken, or power usage to explore the search space until a desired clinical outcome is reached (assuming multiple outcomes with the same or comparable clinical response); among others.

In an example, the optimization criterion includes the clinical response values 538 exceeding a threshold value or falling into a specified value range, indicating a satisfactory therapeutic outcome has reached. Depending on how the clinical response values are computed, one or more optimal base stimulation settings may be determined. For example, the clinical response values may be computed using a single response effect (e.g., one of bradykinesia, tremor, or rigidity). Accordingly, three optimal base stimulation settings may be generated: a first optimal base stimulation setting (BSS₁) corresponding to a bradykinesia score exceeding a threshold, a second optimal base stimulation setting (BSS₂) corresponding to a tremor score exceeding a threshold, and a third optimal base stimulation setting (BSS₃) corresponding to a rigidity score exceeding a threshold. In another example, the clinical response values may be a composite score computed as a weighted combination of multiple clinical effects, such as a %*bradykinesia+b %*tremor+c %*rigidity. Accordingly, a fourth optimal base stimulation setting (BSS₄) may be generated, corresponding to the composite clinical response score exceeding a threshold. In some examples, the stimulation setting optimization may be performed in an in-clinic programming session such during implantation or revision of a DBS system or device follow-up.

The optimal base stimulation settings (e.g., BSS₁ through BSS₄), may be stored in the memory 528. In an example, a stimulation setting, along with the corresponding unique clinical response indicator (e.g., weighted combination of clinical effects with unique weight factors) form a stimulation program 539, which may also be stored in the memory 404. Each stimulation programmed may be associated with, or tagged by, one or more unique clinical response indicators. In some examples, the clinical response values 538 may be weighted according to the time at which the test took place.

In various examples, the stimulation parameter control system 531 may be executed on its own and is not connected to a controller. In such instances it may be used to merely determine and suggest programming parameters, sequences of blocks, visualize a parameter space, test potential parameters, etc.

The process of searching for a stimulation setting (e.g., an electrode configuration and/or stimulation parameter values) typically involves significant computation and time, especially when electrode configuration involves segmented electrodes in a directional lead. If testing all possible settings in the entire parameter space (including electrode configurations, combinations of stimulation parameter values and neurostimulation patterns of stimulation blocks) is done as comprehensively as possible, stimulation would need to be provided to the patient for each possible setting, which may end up with a burdensome and time-consuming programming session. Because practically a programming session may only last a few hours, only a fraction of possible electrode configuration and stimulation parameter combinations may reasonably be tested and evaluated. To reduce the time taken and to improve the efficiency of stimulation setting optimization process, a reduced or restricted electrode configuration and parameter search space may be used. By applying limitations or constraints to the electrode configurations and parameter values, the restricted search space may include a subset of electrodes (e.g., a subset of ring electrodes and/or a subset of segmented electrodes on a lead) that are selected as active electrodes for delivering stimulation, and values or value ranges for one or more stimulation parameters (e.g., a range of current amplitude ranges for an active electrode). Stimulation setting optimization, when performed within such a search space, may be more efficient and cost-effective than searching through the entire parameter space for one or more optimal base stimulation settings such as BSS₁-BSS₄ as discussed above. Identifying a search space may involve identifying a variable ramp for testing increasing adjustable parameter values.

The search space identifier 534 may automatically determine a search space 540 for a stimulation lead at a neural target, such as a region in a brain hemisphere, by imposing certain limitations or constraints on the electrode configurations and/or parameter values or value ranges. In an example, the search space 540 may be determined based on spatial information of the lead, such as lead positions with respect to neural targets, which may be obtained from imaging data of the lead and patient anatomy. Additionally, or alternatively, the search space 540 may be determined based on physiological information such as physiological signals sensed by the electrodes at their respective tissue contact locations. The physiological information may include patient clinical responses to stimulation. In some examples, prior knowledge about patient medical condition, health status, DBS treatment history may be used to determine the search space 540. In an example, the search space identifier 534 may exclude those electrodes on the lead that are out of a region of interest, such that the search space includes only those electrodes within the target of interest. One or more stimulation parameters may be restricted to take certain values or within value ranges. For example, the restricted search space may include certain electrode positions and value ranges for stimulation current amplitude, frequency, or pulse width. The feedback control logic 532 may determine one or more optimal base stimulation settings (e.g., BSS₁-BSS₄) by searching through the identified search space 540. The identified search space 540 may be stored in the memory 528.

The feedback control logic 532 may include a machine learning engine 541 that may facilitate the stimulation parameter control system 531 (or a user of the system) to explore the search space in order to choose values for programming the DBS controller 533. The machine learning engine 541 may employ supervised or unsupervised learning algorithms to train a prediction model, and use the trained prediction model to predict patient clinical responses to an untested stimulation setting (e.g., untested stimulation parameter values or untested electrode configurations), or to estimate or predict stimulation parameters values or electrode configurations that, when provided to the DBS controller 533 to deliver stimulation accordingly to the patient 536, would produce desired or improved clinical responses. Examples of the learning algorithms include, for example, Naive Bayes classifiers, support vector machines (SVMs), ensemble classifiers, neural networks, Kalman filters, regression analyzers, etc. The machine learning engine 541 may build and train a prediction model using training data, such as stimulation parameter values and corresponding patient clinical responses. The training data may be acquired from a training session such as performed in a clinic. Additionally, or alternatively, the training data may be obtained from historical data acquired by the stimulation parameter control system 531. With its learning and prediction capability, the machine learning engine 541 may aid a user (e.g., a clinician) in exploring the stimulation parameter space more effectively and more efficiently to produce results that are optimal, desired, or acceptable.

In some examples, the machine learning engine 541 may use imaging data to inform the choice of the next set of values, which may be used when the algorithm finds itself in a region of parameter space for which the clinical responses are not substantially affected by the changes in the stimulation parameters, and the choice of next step is not apparent from the patient response alone. Imaging data that provides information about the location of the lead in the patient's brain along with priors informing the algorithm of which directions may be better choices for the next step could lead to faster convergence.

In some examples, the machine learning engine 541 may determine expected outcomes for parameter values or neurostimulation patterns that have not yet been tested based upon what the machine learning engine 541 has “learned” thus far, and provide a recommendation for a next set of values to test. Here, testing refers to the iterative testing required to find an optimal stimulation setting for configuring the DBS controller 533. The recommendation for a next set of values or patterns to test is based upon which of the determined expected outcomes meet a set of designated (determined, selected, preselected, etc.) criteria (e.g., rules, heuristics, factors, and the like). For example, rules considered may include such factors as: the next set of values may not be one of the last 10 settings tested or may not be too close to previously tested setting. Accordingly, the feedback control logic 532 with its machine learning engine 541 is used to systematically explore the stimulation parameter space based upon what it has learned thus far and (optionally) different rules and/or heuristics that contribute to achieving optimal outcomes more efficiently.

The process for determining expected outcomes for parameter values that have not yet been tested may involve use of other data for machine learning. For example, data from other programming sessions for the same patient as well as from other patients may be used to train the machine learning engine 541. In some examples, no prior data may be used. In this case, the machine learning engine 541 may use data learned from this patient only in one particular setting. In other examples, data from the same patient but from previous sessions may be used. In some examples all patient data from all sessions may be used. In some examples all patient data utilizing lead location information (knowledge of lead location in space relative to anatomy) may be used. Different other combinations are also possible.

In order to use this data for machine learning purposes, the data may first be cleansed, optionally transformed, and then modeled. In some examples, new variables are derived, such as for use with directional leads, including central point of stimulation, maximum radius, spread of stimulation field, or the like. Data cleansing and transformation techniques such as missing data imputation and dimension reduction may be employed to prepare the data for modeling.

The machine learning engine 541 may determine how best a predicted outcome meets the optimal outcome metrics. Various optimization techniques may be used, examples of which may include but are not limited to: optimization algorithms and estimation procedures used to fit the model to the data (e.g., gradient descent, Kalman filter, Markov chain, Monte Carlo, and the like); optimization algorithms reformulated for search (e.g., simulated annealing); spatial interpolation (e.g., kriging, inverse distance weighting, natural neighbor, etc.); supplementary methods that aid the optimization process (e.g., variable selections, regularization, cross validation, etc.); other search algorithms (e.g., golden-section search, binary search, etc.). Using any of these techniques, the machine learning engine 541 may decide whether a particular predicted outcome for a set of stimulation parameter values is the fastest sufficing outcome, the best possible clinical outcome, or the optimal outcome with least battery usage, for example.

The feedback may be provided directly by the patient 536, entered by an observer such as a clinician (not shown), or may be provided by means of a sensor 537 associated with and in physical, auditory, or visual contact with the patient 536. Examples may include, but are not limited to, accelerometers, microphones, and cameras. In an example, the sensor 537 may be included in a wearable device associated with patient 536, such as a smart watch. In an example where the feedback may be monitored automatically or semi-automatically, such as with use of sensor 537, it may not be necessary for a clinician or other observer to be present to operate the stimulation parameter control system 531. Accordingly, in such examples a user interface may not be present in system 531.

In some examples, the stimulation parameter control system 531 may determine one or more optimal base stimulation settings using predicted clinical responses for untested stimulation parameter values or untested electrode configurations without actually delivering stimulation. Such base stimulation settings are referred to as estimated or predicted base stimulation settings, to distinguish from the tested base stimulation settings (e.g., BSS₁-BSS₄) that are based on the tested clinical response (either reported by the patient or measured by a sensor) to actually delivered stimulation. For examples, based on the “tested” base stimulation settings BSS₁-BSS₄, the stimulation parameter control system 531 may estimate an optimal base stimulation setting associated with a composite clinical response defined as x %*bradykinesia+y %*tremor+z %*rigidity, or simply denoted by the weight factors (x %, y %, z %).

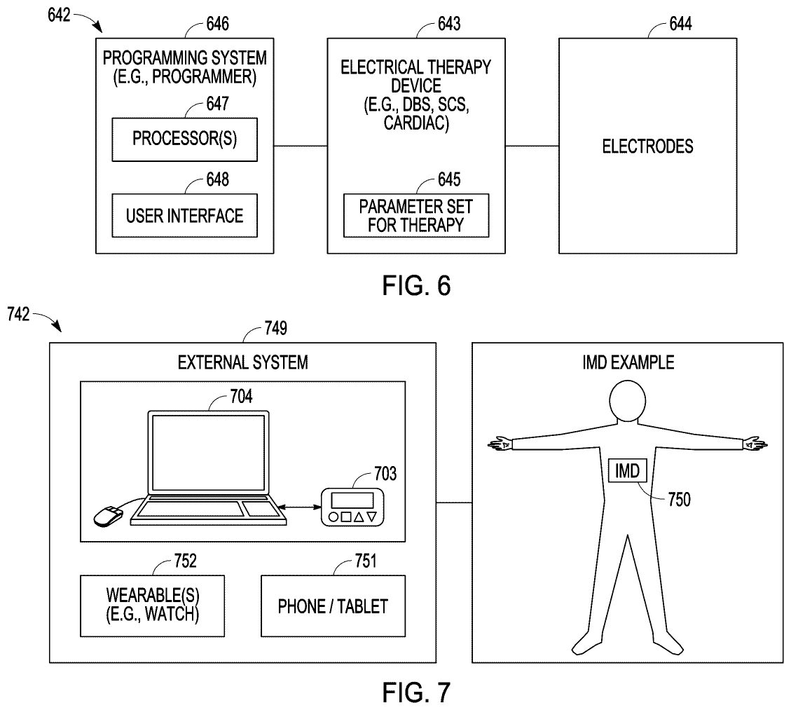

FIG. 6 illustrates, by way of example, an example of an electrical therapy-delivery system. The illustrated system 642 includes an electrical therapy device 643 configured to deliver an electrical therapy to electrodes 644 to treat a condition in accordance with a programmed parameter set 645 or sequence of parameter sets to provide a patterned neurostimulation for the therapy. The system 642 may include a processing system 646 that may include one or more processors 647 and a user interface 648, which may be used to program and/or evaluate the parameter set(s) used to deliver the therapy. The illustrated system 642 may be a DBS system for treating a movement disorder, such as has been illustrated and discussed with respect to FIGS. 1-5, and/or a system for monitoring the movement disorder.

In some embodiments, the illustrated system 642 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

FIG. 7 illustrates, by way of example and not limitation, the electrical therapy-delivery system of FIG. 6 implemented using an implantable medical device (IMD). The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a may further include remote control device 703, similar to RC 103 in FIG. 1, configured for use by the patient to communicate with and program the neuromodulator. For example, the remote control device 703 may allow the patient to turn a therapy on and off and/or may allow the patient to adjust patient-programmable parameter(s) of the plurality of stimulation parameters. FIG. 7 illustrates an IMD 750, although the monitor and/or therapy device may be an external device such as a wearable device. The external system 749 may include a network of computers, including computer(s) remotely located from the IMD 750 that are capable of communicating via one or more communication networks with the programmer 704 and/or the remote control device 703. The remotely located computer(s) and the IMD 750 may be configured to communicate with each other via another external device such as the programmer 704 or the remote control device 703. The remote control device 703 and/or the programmer 704 may allow a user (e.g., patient and/or clinician or rep) to answer questions as part of a data collection process. The external system 749 may include personal devices such as a phone or tablet 751, wearables such as a watch 752, sensors or therapy-applying devices. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. The phone and/or tablet may include camera(s), microphone(s), accelerometer(s) or other sensors that can be used to provide feedback.

A pattern of neurostimulation pulses may be defined by a stimulation program including individually programmable program building blocks. Examples of the individually programmable program building blocks include individually programmable pulses, blocks, and sequences. The individually programmable pulses can each be defined using programmable parameters such as pulse waveform (e.g., monophasic, biphasic, multiphasic, passive recharge, active recharge, or burst), pulse amplitude, pulse width, and/or interphasic interval. The individually programmable blocks can each be a stimulation block during which one or more neurostimulation pulses are delivered or a non-stimulation block during with no pulse is delivered. The stimulation block may be defined using programmable parameters such as pulse amplitude, pulse width, pulse rate, number of pulses or block duration, and/or stimulation field (electrode configuration specifying active electrodes and/or fractionalization). The stimulation blocks may have functions that describe modulation of one or more of the programmable parameters over time. For example, a stimulation block may be defined with a sinusoidal modulation of a pulse amplitude. The non-stimulation block may be defined using a programmable parameter of delay duration (duration of the non-stimulation block). The individually programmable sequences may each include one or more blocks. In various embodiments, a sequence may be composed by creating one or more blocks and/or selecting one or more blocks from stored (pre-configured) and/or imported (e.g., from another system/user) blocks, arrange the blocks in a temporal order (e.g., a random, pseudorandom, or fixed order). The number of successive repetitions of each block in the sequence may be programmable.

A stimulation program may be composed to include one or more sequences, one or more blocks, and/or one or more pulses arranged in a programmable temporal order. The stimulation program may include one or more sequences and/or other stimulation patterns (e.g., pulses, burst of pulses, blocks) in a random, pseudorandom, or fixed order. One or more stimulation programs can be scheduled to be delivered to the patient as the therapy. In various embodiments, a neurostimulation system may be used to compose the stimulation programs, including their building blocks, and to schedule delivery of neurostimulation according to one or more of the composed stimulation programs for applying the therapy.

FIG. 8 illustrates, by way of example and not limitation, an embodiment of a stimulation programming circuit 853 for use in a neurostimulation system. The stimulation programming circuit 853 may include composition circuitry 854 and scheduling circuitry 855. The composition circuitry (also referred to as the composer) 854 may be used to compose a pattern of neurostimulation pulses, including one or more programs and program building blocks (e.g., pulses, blocks, and/or sequences). The scheduling circuitry 855 may schedule delivery of neurostimulation according to the composed pattern (e.g., one or more programs) of neurostimulation pulses. In various embodiments, the composition circuitry 854 and the scheduling circuitry 855 operate with a user interface that may include a presentation device and a user input device to allow for user control in the composition of the pattern of neurostimulation pulses. The composition circuitry 854 may include program building block editors 856 for creating, editing, and/or importing each building block of a stimulation program and a program editor 857 for creating, editing, and/or importing the stimulation program. The program building block editors 856 may include a pulse editor 858 for creating, editing, and/or importing each pulse, a block editor 859 for creating, editing, and/or importing each block, and a sequence editor 860 for creating, editing, and/or importing each sequence. The program editor 857 may create each program using various combinations of the created, edited, and/or imported program building blocks arranged in a temporal order according to which the neurostimulation is to be delivered.

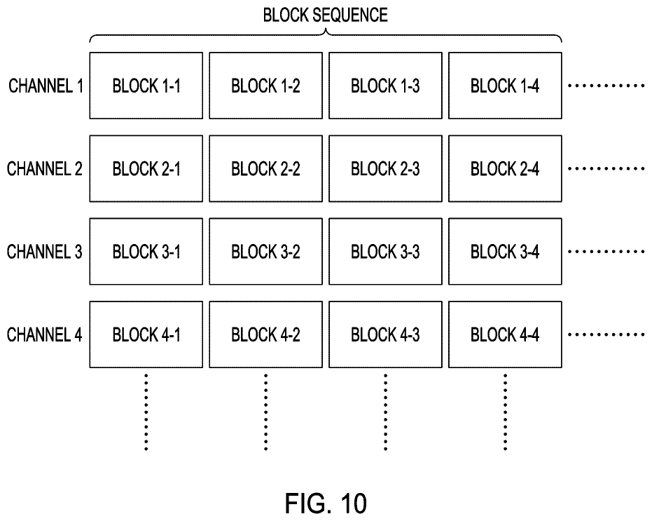

FIG. 9 illustrates, by way of example and not limitation, an embodiment of a system 961 for delivering neurostimulation such as a DBS therapy. The system 961 may include a stimulation device 962, a programming control circuit 963, and a stimulation programming circuit 964. For example, the stimulation device 962 may be implemented in an implantable neurostimulator. The programming control circuit 916 may be implemented in clinical programmer, a remote control or other external device.

The stimulation device 962 may be connected to electrodes and deliver the neurostimulation to a patient using the electrodes. The programming control circuit 963 may generate information for programming stimulation device 962 to deliver the neurostimulation according to a pattern of neurostimulation pulses. The stimulation programming circuit 964 may determine the pattern of neurostimulation pulses. In various embodiments, the stimulation programming circuit can determine the pattern of neurostimulation pulses to be applied in a therapy automatically, semi-automatically by interacting with the user, and/or manually based on inputs from the user.

The stimulation programming circuit 964 may be configured to receive one or more goal options, to select one or more programmability options associated with the received one or more goal options, to receive programming information required by the selected one or more programmability options, and to determine the pattern of neurostimulation pulses for the selected one or more goal options using the received programming information. The one or more goal options can be selected from a plurality of goal options each including at least one goal for the DBS therapy (e.g., a therapeutic objective, or a device power efficiency). The one or more programmability options can be selected from a plurality of programmability options each allowing one or more aspects (e.g., types and/or value ranges of stimulation parameters) of the pattern of neurostimulation pulses and/or the delivery of the neurostimulation according to the pattern of neurostimulation pulses to be programmable. The receive programming information required by each selected programmability option can include, for example, values of stimulation parameters designated to be programmable under that programmability option.

In various embodiments, the stimulation control circuit 963 and the stimulation programming circuit 964 are included in a programming device for programming stimulation device 962. For example, when an implantable stimulator includes the stimulation device 962, the external programming device may be configured for programming control circuit 963 to include the programming control circuit 963 to include stimulation control circuit 964. A user interface can be configured to include a composer (e.g., using stimulation control circuit functioning with presentation device and user input device) to compose one or more patterns of neurostimulation pulses and to schedule delivery of one or more patterns of neurostimulation pulses. Each pattern of neurostimulation pulses, and/or its building blocks, can be edited and/or imported using the composer.

FIG. 10 illustrates, by way of example and not limitation, block sequences for four timing channels. Each of the channels has a block sequence corresponding to the channel's row in the table. For example, the first timing channel corresponds to Block 1-1, Block 1-2, Block 1-3 and Block 1-4. Similarly, the second timing channel corresponds to Block 2-1, Block 2-2, Block 2-3 and Block 2-4. Each block represents its own modulation parameter set, including an electrode configuration for that modulation parameter set. Thus, different blocks may correspond to different modulation fields that have different target pole(s), as the electrode configurations may have different activated electrodes, and/or different fractionalized values. These different modulation fields may target completely different volumes of tissue, or may generally target the same volume of tissue but using different polarities and modulation field shapes to create different field orientations. The present subject matter is not limited to four channels as the number of channels may be more or less than four, and is not limited to four blocks per channel, as the number of blocks per channel may be more or less than four blocks. Furthermore, the timing of the blocks (e.g., start time, stop time, duration, inter-block intervals, etc.) may be independently controlled for each of the timing channels.

Programmable settings for the pulse waveform may include a pulse amplitude, a pulse width, a pulse frequency, a pulse train duration, a pulse-to-pulse duty cycle, a pulse train to pulse train duty cycle (stimulation ON/OFF), and a stimulation schedule (e.g., programmable start and/or stop times, such as but not necessarily a calendar-based schedule). The programmable settings may further include controlling which of a plurality of electrodes are active and which are off, the polarity of each active electrode (which active electrode(s) are anode(s) and which are cathode(s), and the contributions (e.g., electrode fractionalization) of total energy delivered to individual one(s) of the anode(s) and individual one(s) of the cathode(s). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. Controlling the individual contributions by individual electrodes adjusts the location and shape of the stimulation field, to modulate different combinations of neural elements.

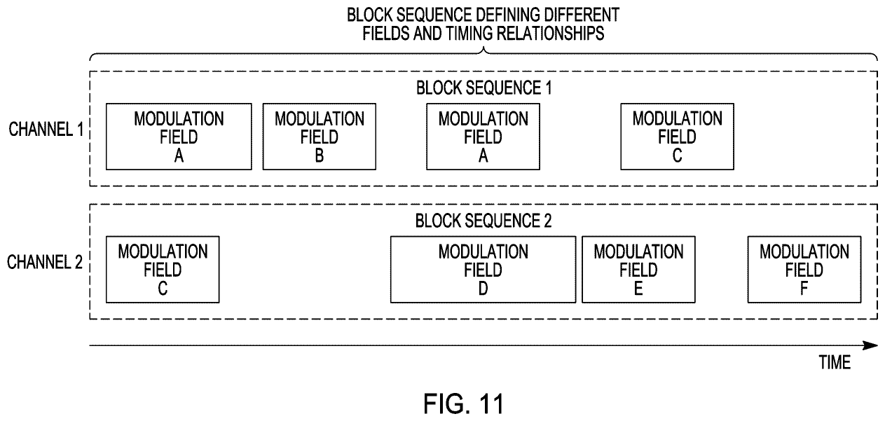

FIG. 11 illustrates, by way of example and not limitation, block sequences for different channels with illustrated timing relationships between the blocks in those sequences. The different fields (“A”, “B”, “C”, D ”, E” and “F”) may define different patterns of neurostimulation and/or different spatial modulation fields that may be the result of different fractionalizations of the anodic and cathodic contributions from active electrodes. The same field may intermittently occur in a sequence. The timing relationships may be programmed by the user so that the different fields will have different start time, stop time, duration, etc. The timing relationships may be based on an absolute time (system clock), or may be based on relative timing relationships between blocks in the same or different timing channels such as, by way of example and not limitation, a delay after another block starts or delay after another block ends. The timing relationships may include whether a sequence repeats and timing between repeats of the sequence.

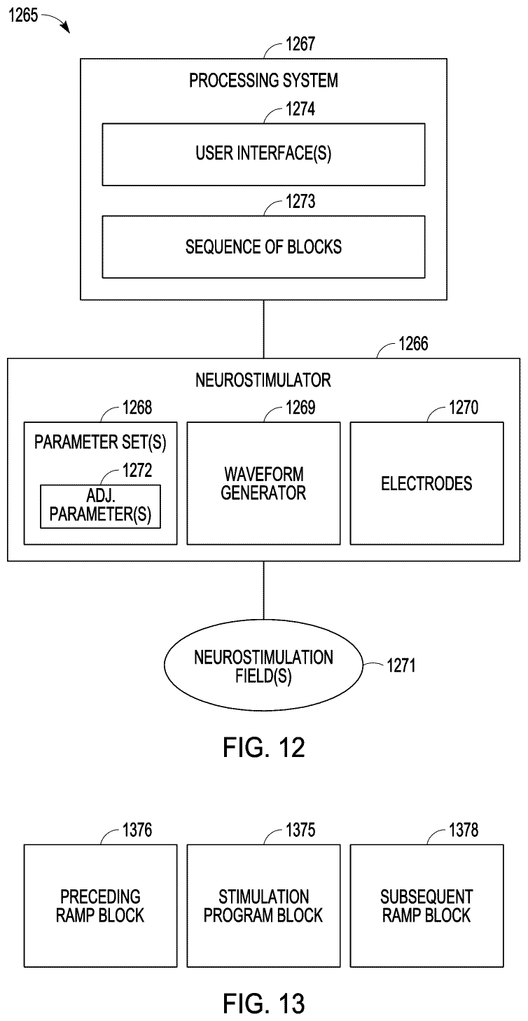

FIG. 12 illustrates, by way of example and not limitation, a system configured to deliver neurostimulation according to sequences of blocks. The illustrated system 1265 may include a neurostimulator 1266 and a processing system 1267. The neurostimulator 1266 may be programmed or otherwise configured with at least one parameter set. The neurostimulator 1266 may use the parameter set(s) 1268 along with a waveform generator 1269 to generate a neurostimulation waveform signal to an electrode configuration. The parameter set(s) may define the electrode configuration, including the active electrodes from a plurality of available electrodes 1270 and the polarity of each individual ones of the active electrodes. That is, at a given time on a given channel, each active electrode may be configured as an anode or cathode. The parameter set(s) may further define electrode fractionalizations at a given time on a given channel, which refers to the contributions of each active anodic electrode to the overall anodic energy and the contributions of each active cathodic electrode to the overall cathodic energy. Thus, the neurostimulator 1266 may be used to create the neurostimulation field(s) 1271 by delivering electrical energy according to a set of values for a stimulation parameter set which may include adjustable parameter(s) 1272 that have one set of value(s) at one time and other sets of value(s). The processing system 1267 may be configured to create a sequence of blocks 1273 to determine the neurostimulation field(s) 1271 provided by the neurostimulator 1266. The processing system 1267 may include user interface(s) 1274 which may be configured to, among other things, provide user input or control used in the creation of the sequence of blocks. The user interface(s) 1274 may include one or more screens on a display such as a touch screen display. By way of example and not limitation, the user interface(s) may include keyboards or keypads, mouse, pointer, manual switches, knobs, buttons, joysticks or voice command. The processing system 1267 may be configured to communicate with or program the neurostimulator 1266 with the corresponding parameter set(s) 1268 to implement the sequence of blocks 1273 in a timing channel. The processing system 1267 may include remote and/or local systems. For example, the processing system 1267 may include cloud computing, fog computing, and/or edge computing. Cloud computing may include a network of devices or servers connected over the Internet. Cloud computing may have very large storage space and processing capabilities. However, cloud computing can have higher latencies. Fog computing occurs physically closer to the end user compared to centralized data centers. The infrastructure of fog computing may connect end devices with central servers in the cloud. Fog computing may provide lower latency for quicker responses and may use other communication technology other than the Internet. Edge computing is done at the device level. The processing for different functions may be distributed over multiple devices and may be distributed over edge computing, fog computing and cloud computing.

FIG. 13 illustrates, by way of example and not limitation, a stimulation program block 1375 with a preceding ramp block 1376 for ramping toward the stimulation program block 1375 and with a subsequent ramp block 1377 for ramping away from the stimulation program block 1375. With reference to both FIGS. 12 and 13, the sequence of blocks 1273 may include at least one stimulation program block and at least one preceding ramp block 1376 and/or at least one subsequent ramp block 1377. The stimulation program block may provide a portion of the desired neurostimulation pattern for a therapy. The stimulation program block(s) 1375 may be configured to provide the neurostimulation field by delivering electrical energy according to the set of values for the stimulation parameter set. The set of values may include program value(s) for the adjustable parameter(s) 1272. The neurostimulation field delivered according program value(s) correspond to a desired therapy. The stimulation program block may include only one pulse or may include more than one pulse. A stimulation program block may include more than one pulse and each of the at least one adjustable parameter may have one value such that the stimulation delivered by the stimulation program block is a tonic stimulation. The stimulation program block may include more than one pulse and one or more of the at least one adjustable parameter has more than one value such that the stimulation delivered by the program block may be variable.

However, some patients may find some transitions to a stimulation program block, some transitions from a stimulation program block, or some transitions between blocks within the sequence of blocks to be uncomfortable. By way of example, some stimulation program blocks may deliver a relatively high amount of charge over a unit of time (e.g., “neurostimulation dose”), and it may be desirable to have a ramp for transitioning to the high dose or a ramp for transitioning from the high dose. For example, a difference between amplitudes or pulses widths when transitioning from one parameter set to another parameter set may cause discomfort. The ramp block(s) may be used to assist with transitioning between blocks. The ramp block(s) 1376, 1378 may be configured to determine a ramping sequence for changing the parameter values for the adjustable parameter(s). The ramp block(s) may be configured to determine a ramping sequence for changing the parameter values for the adjustable parameter(s). A preceding ramp block 1376 may be configured to change parameter values for the adjustable parameter(s) to ramp toward the program value(s) used in the stimulation program block 1375. A subsequent ramp block 1378 may be configured to change the parameter values for the adjustable parameter(s) to ramp away from the program value(s) used in the stimulation program block 1375.

For example, a processing system may be configured to automatically insert the at least one ramp block into the sequence of blocks based on the at least one program value for the at least one adjustable parameter in the at least one stimulation program block. For example, the adjustable parameter may include an amplitude and the program value for the stimulation program block may be a threshold value for an amplitude. The ramp block may be inserted when the amplitude crosses the threshold. By way of example and not limitation, a user may interact with the user interface(s) to insert a preceding ramp block and/or subsequent ramp block. The blocks may be closely concatenated together, or there may be an intervening time between adjacent blocks.

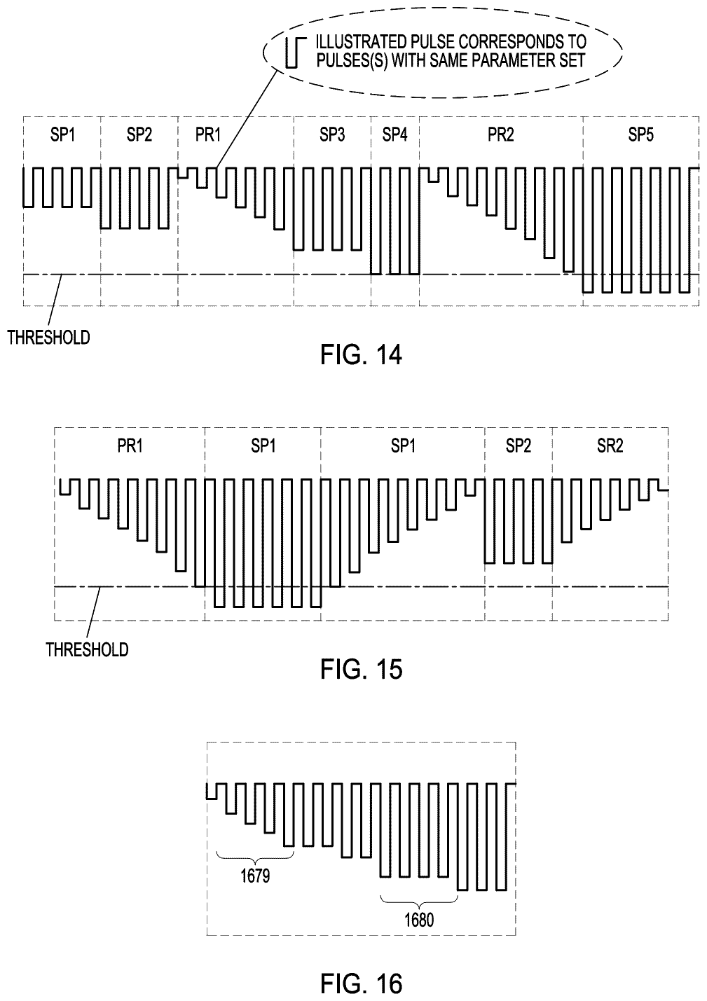

FIG. 14 illustrates, by way of example and not limitation, a sequence of blocks including stimulation program blocks and ramp blocks. The illustration includes a number of pulses within each block. Each pulse may correspond to a single pulse or may correspond to more than one pulse with the same parameter set. The blocks may be closely concatenated together, or there may be an intervening time between adjacent blocks. The illustrated sequence of blocks includes five stimulation program (SP) blocks, labeled SP, SP2, SP3, SP4 and SP5, as well as two preceding ramp (PR) blocks, labeled PR1 and PR2. Thus, stimulation program blocks SP1 and SP2 may be concatenated without an intervening ramp block. However, a user may choose to insert a preceding ramp block PR 1 before stimulation program block SP3. Stimulation program blocks SP3 and SP4 may be concatenated without an intervening ramp block. Stimulation program block SP5 is illustrated as exceeding a threshold as represented by the dotted line. For example, the threshold may be an amplitude threshold for the amplitude used by the stimulation program block. However, the threshold may include other adjustable parameters such as but not limited to pulse width and frequency or combinations of parameters such as combinations of two or more of amplitude, pulse width and frequency. The system may be configured to automatically insert preceding ramp block PR2 when the threshold is crossed.

FIG. 15 illustrates, by way of example and not limitation, a sequence of blocks including stimulation program blocks and preceding and subsequent ramp blocks. The illustrated sequence of blocks includes two stimulation program (SP) blocks, labeled SP1 and SP2, as well as one preceding ramp (PR) blocks, labeled PR1 and two subsequent ramp (SR) block, labeled as SR1 and SR2. PR blocks and/or SR blocks may be entered by the user via the user interface. PR blocks and/or SR blocks may be automatically inserted into the sequence. For example, specific ramp blocks may be automatically inserted if the SP block satisfies one or more criteria. In the illustrated example, SP1 exceeds a threshold which may trigger the system to automatically insert PR1 and SR1.

FIG. 16 illustrates, by way of example and not limitation, pulse-to-pulse variations that may be implemented in a ramp block. The ramp blocks may, but need not, provide linear changes to the adjustable parameter(s) throughout the ramp block. The specific configuration of the ramp block may be based on criteria of the SP block. A ramp block, or a portion of the ramp block, may include as shown at 1679 a sequence of three pulses corresponding to three parameter values for the adjustable parameter(s). The change between parameter values for adjacent pulses may be the same (e.g., a linear ramp) or may be different (e.g., a nonlinear ramp). A ramp block, or a portion of the ramp block, may include as shown at 1680 a sequence of at least two pulses corresponding to one parameter value for the adjustable parameter(s). Thus, the ramp may include a stepped progression as the adjustable parameter value(s) move toward or away from the programmed values in the SP block.

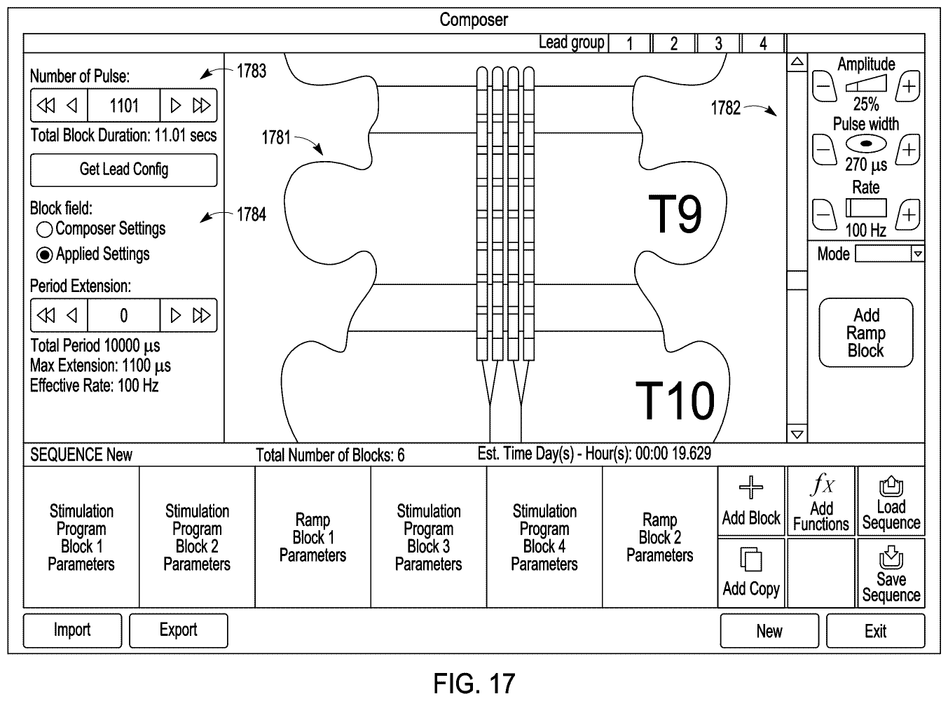

FIG. 17 illustrates, by way of example and not limitation, a user interface for programming a sequence of ramp blocks. The user interface may include a region 1781 displaying a representation of an anatomical region and lead(s)/electrode(s) that are available to create neuromodulation field(s). Some embodiments may display representations of the field(s) based on parameter set(s). The user interface may include a region 1782 to program pulse parameter(s) such as amplitude, pulse width and pulse rate (frequency) for a block, a region 1783 to program the number of pulses in a block or a duration for the block, and a region 1784 that may be used to evaluate settings before they are programmed into the neurostimulator or settings that are currently programmed in the neurostimulator. The illustrated user interface enables both stimulation program blocks and ramp blocks to be configured. A copy of a block or blocks may be added. A function may be used to control the changes in the adjustable parameter(s). User controls may be available to create, save and load sequences of blocks. The ramp blocks may be preprogrammed blocks or may be user-defined ramp blocks.

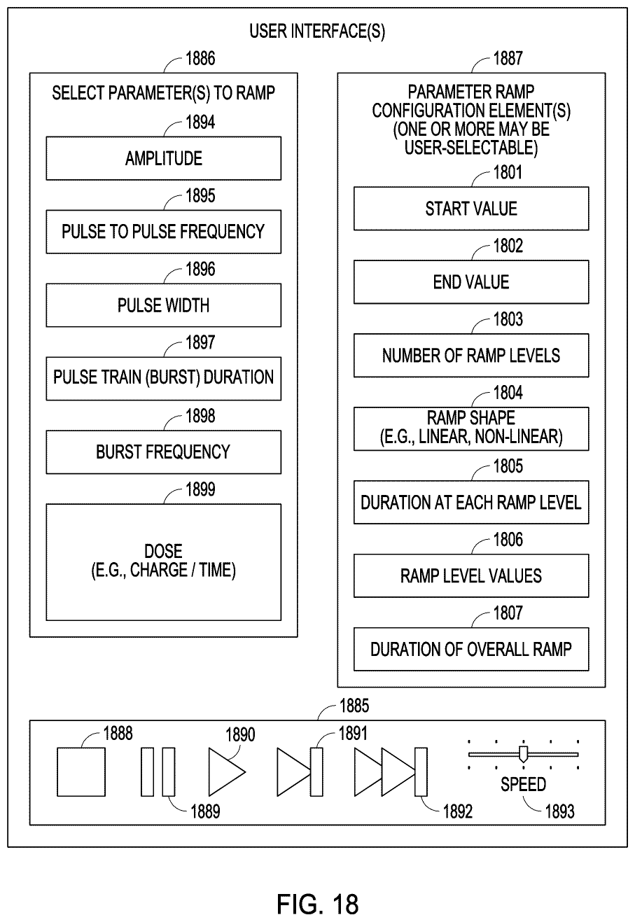

FIG. 18 illustrates, by way of example and not limitation, features that may be implemented in at least one user interface. The user interface(s) may include keyboards or keypads, mouse, pointer, manual switches, knobs, buttons, joysticks or voice command. User input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard.

By way of a nonlimiting example, one or more touchscreen display screens may include a ramp control interface 1885, a ramp parameter selection interface 1886, and a ramp configuration interface 1887. The ramp control interface 1885 may be configured to enable user control of the ramping sequence when the neurostimulator is providing the neurostimulation field according to the ramping sequence. The ramp control interface 1885 may include at least one of a stop element 1888 to provide a stop command to the processing system to stop the ramping sequence, a pause element 1889 to provide a pause command to the processing system to pause the ramping sequence, a play element 1890 to provide a play command to the processing system to normally progress through the ramping sequence, a skip element 1891 to provide a skip command to the processing system to skip to another value in the ramping sequence, a skip ramp element 1892 to provide a skip ramp command to the processing system to skip to an end of the ramping sequence, or a speed control 1893 to provide a speed command to the processing system to change a rate for progressing through the ramping sequence.

The ramp parameter selection interface 1886 may be configured to enable user selection of the adjustable parameter(s) in the ramp block(s) from at least two available adjustable parameters. The available adjustable parameter(s) may include a pulse amplitude 1894, a pulse width 1895, a pulse-to-pulse frequency 1896, a pulse train duration 1897, a burst frequency 1898, and/or a dose 1899. The dose may represent an amount of charge delivered over a period of time. Amplitude and pulse width are two nonlimiting examples of pulse parameters that may affect the dose.

The ramp configuration interface 1887 may be configured to enable user configuration of the ramping sequence. The ramp configuration interface 1887 may allow a user to select or otherwise provide a start value 1801 for the ramping sequence, an end value 1802 for the ramping sequence, a number of ramp levels 1803, a ramp shape 1804, a duration 1805 for at least some of the ramp levels, values 1806 for at least some of the ramp levels, or an overall ramp duration 1807. The ramp configuration interface 1887 may be configured to enable user configuration of the ramp shape 1804 for the ramping sequence. The ramp shapes may include linear or non-linear ramp shapes. The user may select a function or shape for the ramp. Comfort may be a factor for determining an appropriate ramp speed. For example, a fast ramp may be used if the stimulation is imperceptible and a slow ramp may be used if the stimulation is perceptible and nearing a level of discomfort. Patients may become exhausted if levels of significant discomfort are often reached.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, examples in which only those elements shown or described are contemplated. Moreover, examples using combinations or permutations of those elements shown or described are also contemplated.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media (referred to herein as computer readable medium), such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine” may include at least one processor/controller, including one processor/controller to implement all of the instructions, at least two processors/controllers where one processor/controller operates on some of the instructions and other processor(s)/controller(s) operate on other instructions, or at least two processors/controllers where each processor/controller is capable of operating on the same instructions. Thus, for example, distributed systems or systems with shared resources are contemplated.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
creating a sequence of blocks to determine a neurostimulation field provided by a neurostimulator, wherein the sequence of blocks includes:
at least one stimulation program block configured to provide the neurostimulation field by delivering electrical energy according to a set of values for a stimulation parameter set that includes at least one adjustable parameter, wherein the set of values includes at least one program value for the at least one adjustable parameter; and
at least one ramp block, including at least one of:
a preceding ramp block configured to change parameter values for the at least one adjustable parameter to ramp toward the at least one program value; or
a subsequent ramp block configured to change the parameter values for the at least one adjustable parameter to ramp away from the at least one program value; and
programming the neurostimulator to provide the neurostimulation field according to the sequence of blocks.

2. The method of claim 1, wherein the at least one adjustable parameter includes at least one of:
a pulse amplitude;
a pulse width;
a pulse-to-pulse frequency;
a pulse train duration;
a burst frequency; or
a dose parameter.

3. The method of claim 2, wherein the at least one adjustable parameter includes the pulse amplitude and the at least one ramp block only adjusts the pulse amplitude.

4. The method of claim 1, wherein the stimulation program block includes more than one pulse and each of the at least one adjustable parameter has one value.

5. The method of claim 1, wherein the stimulation program block includes more than one pulse and one or more of the at least one adjustable parameter has more than one value.

6. The method of claim 1, wherein the stimulation program block includes one pulse and the at least one adjustable parameter includes at least one of a pulse amplitude or a pulse width for the one pulse.

7. The method of claim 1, wherein the creating the sequence of blocks includes automatically inserting the at least one ramp block into the sequence of blocks based on the at least one program value for the at least one adjustable parameter in the at least one stimulation program block.

8. The method of claim 1, wherein the creating the sequence of blocks includes receiving user input and inserting the at least one ramp block based on the received user input.

9. The method of claim 1, wherein the at least one ramp block includes a sequence of three pulses corresponding to three parameter values for the at least one adjustable parameter.

10. The method of claim 1, wherein the at least one ramp block includes a sequence of at least two pulses corresponding to one parameter value for the at least one adjustable parameter.

11. The method of claim 1, further comprising using the neurostimulator to provide the neurostimulation field according to a ramping sequence and providing a ramp control interface to enable user control of the ramping sequence when the neurostimulator is providing the neurostimulation field according to the ramping sequence.

12. The method of claim 11, wherein the ramp control interface includes at least one of:
a stop element to provide a stop command to a processing system to stop the ramping sequence;
a pause element to provide a pause command to the processing system to pause the ramping sequence;
a play element to provide a play command to the processing system to normally progress through the ramping sequence;
a skip element to provide a skip command to the processing system to skip to another value in the ramping sequence;
a skip ramp element to provide a skip ramp command to the processing system to skip to an end of the ramping sequence; or
a speed control to provide a speed command to the processing system to change a rate for progressing through the ramping sequence.

13. The method of claim 1, including providing a ramp parameter selection interface configured to enable user selection of the at least one adjustable parameter in the at least one ramp block from at least two available adjustable parameters, wherein the at least two available adjustable parameters include two or more of:
a pulse amplitude;
a pulse width;
a pulse-to-pulse frequency;
a pulse train duration;
a burst frequency; or
a dose.

14. The method of claim 1, including providing a ramp configuration interface configured to enable user configuration of the ramping sequence, including at least one of:
a start value for the ramping sequence;
an end value for the ramping sequence;
a number of ramp levels;
a duration for at least some of the ramp levels;
values for at least some of the ramp levels;
an overall ramp duration; or
a ramp shape.

15. The method of claim 14, wherein the ramp configuration interface is configured to enable user configuration of the ramp shape for the ramping sequence, including user configuration of a non-linear ramp shape.

16. A non-transitory machine-readable medium including instructions, which when executed by a machine, cause the machine to perform a method, comprising:
creating a sequence of blocks to determine a neurostimulation field provided by a neurostimulator, wherein the sequence of blocks includes:
at least one stimulation program block configured to provide the neurostimulation field by delivering electrical energy according to a set of values for a stimulation parameter set that includes at least one adjustable parameter, wherein the set of values includes at least one program value for the at least one adjustable parameter; and
at least one ramp block, including at least one of:
a preceding ramp block for changing parameter values for the at least one adjustable parameter to ramp toward the at least one program value; or
a subsequent ramp block for changing the parameter values for the at least one adjustable parameter to ramp away from the at least one program value; and
programming the neurostimulator to provide the neurostimulation field according to the sequence of blocks.

17. A system, comprising:
a neurostimulator configured to provide a neurostimulation field by delivering electrical energy according to a set of values for a stimulation parameter set, wherein the stimulation parameter set includes at least one adjustable parameter; and
a processing system configured to create a sequence of blocks to determine the neurostimulation field provided by the neurostimulator, wherein the sequence of blocks includes:
at least one stimulation program block configured to provide the neurostimulation field by delivering electrical energy according to the set of values for the stimulation parameter set, wherein the set of values includes at least one program value for the at least one adjustable parameter; and
at least one ramp block configured to determine a ramping sequence for changing the parameter values for the at least one adjustable parameter, including at least one of:
a preceding ramp block configured to change parameter values for the at least one adjustable parameter to ramp toward the at least one program value; or
a subsequent ramp block configured to change the parameter values for the at least one adjustable parameter to ramp away from the at least one program value,
wherein the neurostimulator is configured to provide the neurostimulation field according to the sequence of blocks.

18. The system of claim 17, wherein the processing system is configured to automatically insert the at least one ramp block into the sequence of blocks based on the at least one program value for the at least one adjustable parameter in the at least one stimulation program block.

19. The system of claim 17, wherein the processing system includes a user interface configured to receive user input and the processing system is configured to insert the at least one ramp block based on the received user input.

20. The system of claim 17, wherein the processing system further includes a ramp configuration interface configured to enable user configuration of the ramping sequence, including at least one of:
a start value for the ramping sequence;
an end value for the ramping sequence;
a number of ramp levels;
a duration for at least some ramp levels;
values for at least some ramp levels;
an overall ramp duration; or
a ramp shape.
